(19) United States
(12) Patent Application Publication
(10) Pub. No.: US 2020/0337654 A1
(43) Pub. Date: Oct. 29, 2020

(54) Radiation Device for Patients

(71) Applicant: BEC GmbH, Pfullingen (DE)

(72) Inventor: Matthias Buck, Reutlingen (DE)

(21) Appl. No.: 16/857,205

(22) Filed: Apr. 24, 2020

(30) Foreign Application Priority Data
Apr. 27, 2019 (DE) 20 2019 001 877.8

(51) Int. Cl. A61B 6/04; B25J 9/04; A61B 6/03

(52) U.S. Cl. CPC A61B 6/0407; A61B 6/032; A61B 6/0464; B25J 9/042

(57) ABSTRACT

A patient radiation device has a patient positioning device with a patient receptacle, wherein the patient positioning device is able to carry out at least a horizontal movement of the patient receptacle. A radiation source is provided. An imaging device with an imaging unit is provided to produce an image in an imaging plane. A linear movement device is provided to move the imaging unit along a vertical movement axis. The imaging unit is able to perform a rotation about a horizontally arranged axis of rotation so that imaging can be performed on patients that are seated and on patients that are lying on a table.

### BACKGROUND OF THE INVENTION

The invention relates to a radiation device for patients comprising a patient positioning device, a radiation source, and an imaging device, wherein the patient positioning device comprises at least one patient receptacle, wherein the patient positioning device is configured to perform at least a horizontal movement of the patient receptacle, wherein the imaging device comprises an imaging unit for generating an image in an imaging plane, and wherein the imaging device comprises a linear movement device for movement of the imaging unit along a vertical movement axis.

Radiation devices for patients are known that are provided with a radiation source, an imaging device, as well as a patient positioning device are known. The imaging device is usually employed in order to record immediately prior to the radiation treatment the precise position of the tissue to be irradiated in the patient. By means of the patient radiation device, the patient is initially positioned at the imaging device and subsequently at the radiation source. The imaging unit produces usually an image in an imaging plane. In order to image a larger volume, the imaging unit is moved during imaging, for example, in vertical direction. The patient radiation device is stationary during imaging in known patient radiation devices. With such a vertically movable imaging unit, imaging of seated patients can be carried out very well.

The invention has the object to provide a patient radiation device of the aforementioned kind that is of a simple configuration and can be used in various ways.

### SUMMARY OF THE INVENTION

In accordance with the invention, this is achieved in that the imaging unit is rotatable about a horizontally arranged axis of rotation.

The invention thus provides that the radiation device for patients (patient radiation device) comprises an imaging unit that is rotatable about an horizontally arranged axis of rotation. In this way, a patient in a lying position can also be subjected to imaging with the imaging device when the imaging unit is arranged such that the imaging plane is vertically oriented and the patient during imaging is moved horizontally relative to the imaging unit. For the horizontal movement of the patient relative to the imaging unit, preferably the patient positioning device is utilized. A linear movement device which moves the imaging unit in horizontal direction can thus be eliminated so that a simple configuration of the system results. Due to the rotatability of the imaging unit in combination with the possibility of a horizontal movement of the patient by means of the patient positioning device, seated patients as well as patients lying down can be subjected to imaging in a simple way.

A linear movement device is to be understood herein as a movement device that is embodied to carry out exclusively a linear movement in one direction. The linear movement device comprises therefore precisely one translatory degree of freedom of movement and no rotatory degree of freedom of movement. The linear movement device is preferably a linear guide system.

Preferably, it is provided that the patient positioning device comprises a robot arm. The robot arm is advantageously movable by means of a second linear movement device along a horizontal movement axis. The robot arm is advantageously fixed to a driven carriage of the second linear movement device. Alternatively, it can also be provided that the robot arm itself can carry out a linear, horizontal movement of the patient receptacle with sufficient precision. In this case, a second linear movement device can then be eliminated.

The robot arm comprises preferably at least six axes of rotation, i.e., six axes that are independent from each other in order to be able to pivot the sections of the robot arm relative to each other. Such a six-axis robot enables free positioning and pivoting of the patient receptacle secured to the robot arm. A precise and quick positioning of the patient is possible. The patient receptacle is secured to the robot arm preferably so as to be detachable. Advantageously, the patient positioning device comprises a patient receptacle in the form of a seat and another patient receptacle in the form of a table which can be alternatively connected to the robot arm.

An advantageous arrangement is provided when the robot arm is arranged suspended from the linear movement device. The linear movement device is preferably secured at the ceiling of the room in which the patient radiation device is arranged and the robot arm is arranged with its support suspended from the linear movement device. In this way, the floor is freely accessible for other devices and a large movement range for the patient receptacle can be obtained without the utilization of the room being restricted by installations on the floor.

The imaging device and the radiation source are preferably separable from each other by a structural protective barrier. Many known imaging devices are radiation-sensitive. The radiation of the radiation source leads to wear and aging of the parts of the imaging device. By means of the spatial separation of the imaging device from the radiation source, the radiation exposure of the imaging device can be minimized. The structural protective barrier is preferably openable so that, between the imaging device on one side and the radiation source on the other side, a passage for the patient receptacle can be formed. Opening the structural protective barrier is preferably done in an automatic fashion. The movement of the patient from the room section in which the radiation source is arranged into the room section which is separated therefrom by the structural protective barrier and in which the imaging device is arranged is preferably realized by means of a combined movement of the robot arm and of the linear movement device.

A simple and pleasing configuration results when the imaging unit is of an annular shape. A patient who is in a lying position is preferably moved during imaging by the patient positioning device relative to the annular imaging unit. In case of a patient who is seated, it is preferably provided that the imaging unit during imaging is moved in vertical direction relative to the patient. At least one linear movement device is advantageously is in the form of a linear guide system. In particular, the first linear movement device and the second linear movement device are linear guide systems.

Preferably, the imaging unit comprises precisely two movement axes, i.e., the vertical movement axis and the axis of rotation. Further movement axes which enable linear movements or rotational movements of the imaging unit in space are preferably not provided. In this way, a simple configuration of the imaging unit is provided.

### BRIEF DESCRIPTION OF THE DRAWING

FIG. 1 is a perspective view of a first room section in which the patient positioning device and the radiation source are arranged.

FIG. 2 is a perspective illustration of a second room section in which the imaging device is arranged.

FIG. 3 is a side view of the patient radiation device in an arrangement prior to imaging of a seated patient.

FIG. 4 is a side view during imaging of a seated patient (not illustrated).

FIG. 5 is a perspective illustration of the patient radiation device during movement of a seated patient from the imaging device to the radiation source.

FIG. 6 is a perspective illustration of the patient radiation device in an arrangement for radiation treatment of a seated patient.

FIG. 7 is a patient radiation device in a side view during transport of a patient in a lying position.

FIG. 8 is a perspective illustration of the second room section during transport of the patient to the imaging device.

FIG. 9 is side view of the patient radiation device during imaging of a patient who is in a lying position.

FIG. 10 is a perspective illustration of the patient radiation device during transport of the patient in lying position from the imaging device to the radiation source.

FIG. 11 is a side view of the patient radiation device in a position during radiation treatment of the patient in lying position.

### DESCRIPTION OF PREFERRED EMBODIMENTS

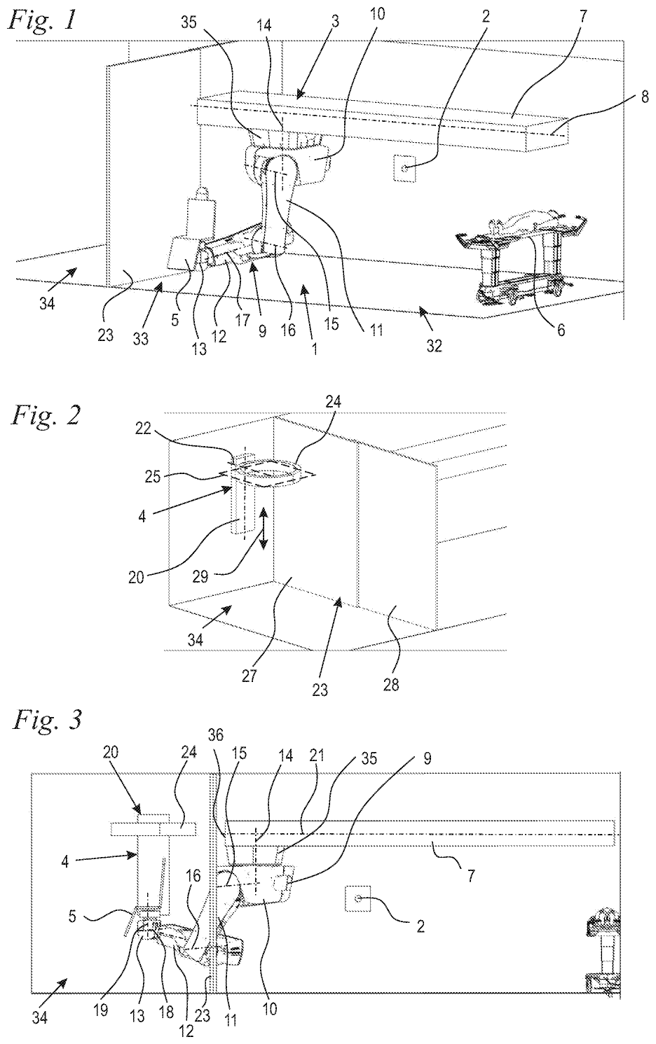

FIG. 1 shows schematically a patient radiation device 1. The patient radiation device 1 is arranged in a room 32 which is divided by a structural protective barrier 23 into two room sections 33 and 34. FIG. 1 shows in detail the first room section 33 in which the radiation treatment of a patient is performed while the second room section 34 is substantially hidden by the structural protective barrier 23. A radiation source 2 opens into the first room section 33. The radiation source 2 can be, for example, a source of protons, neutrons, ions or the like. The radiation source 2 can also generate a photon ray such as an x-ray or gamma ray.

In order to be able to exactly position patients at the radiation source 2, a patient positioning device 3 is provided. The patient positioning device 3 comprises a linear movement device 7 which, in the embodiment, is a linear guide system driven by an electric motor, not illustrated. A robot arm 9 is secured at the linear guide system. The linear guide system comprises a single translatory degree of freedom of movement and no rotatory degree of freedom of movement. The robot arm 9 can be moved by the linear movement device 7 along a horizontally extending movement axis 8. The linear movement device 7 is preferably fixed at the ceiling of the room 32, not illustrated. Instead of the robot arm 9, a differently designed movement device, for example, an arrangement with a plurality of linear movement devices, can be provided also. In the illustration of FIG. 1, a patient receptacle 5 is secured at the robot arm 9 and is embodied as a seat with foot rest, seating surface, and back rest. As an alternative, another patient receptacle 6 embodied as a table, which extends flat and horizontally in the embodiment, can be arranged at the robot arm 9.

The robot arm 9 in the embodiment is designed as a conventional six-axis robot. The robot arm 9 comprises a support 35 which is movable by the linear movement device 7 along the movement axis 8. On the support 35, a base 10 is supported so as to be rotatable about the first axis of rotation 14. The first axis of rotation 14 is arranged vertically and perpendicular to the movement axis 8. At the base 10, a rocker 11 is supported so as to be pivotable about a second axis of rotation 15. The second axis of rotation 15 extends horizontally. At the end of the rocker 11 facing away from the base 10, an arm 12 is arranged and supported to be pivotable about a third axis of rotation 16. The third axis of rotation 16 extends parallel to the second axis of rotation 15. At the end of the arm 12 facing away from the rocker 11, a hand 13 is supported so as to be rotatable about a fourth axis of rotation 17. The fourth axis of rotation 17 extends approximately in the longitudinal direction of the arm 12. The hand 13 comprises a fifth axis of rotation 18 as well as a sixth axis of rotation 19; they are illustrated schematically in FIG. 3. Due to the six axes of rotation 14 to 19, the patient receptacle 5 at the hand 13 can carry out movements in all three spatial directions and rotations about all three axes in space. Since the robot 9 is suspended from the ceiling, the floor is unoccupied.

FIG. 2 shows the second room section 34. In the second room section 34, an imaging device 4, for example, a computed tomography device, is arranged. The imaging device 4 comprises an imaging unit 24 which can comprise in the embodiment an x-ray source, not illustrated, as well as an x-ray detector. By means of the imaging unit 24, an image of the patient in an imaging plane 25 can be produced. The imaging unit 24 is annular and the imaging plane 25 is positioned perpendicular to the central axis of the ring forming the imaging unit 24. The imaging unit 24 is secured at a linear movement device 20. The linear movement device 20 enables movement of the imaging unit 24 in the direction of a vertically arranged movement axis 21. This is illustrated by the double arrow 29 in FIG. 2. The imaging unit 24 is also secured so as to be rotatable about an axis of rotation 22 at the linear movement device 20. The axis of rotation 22 extends horizontally and in the embodiment perpendicular to the wall at which the linear movement device 20 is arranged. The imaging device 4 comprises in the embodiment exclusively two axes of movement, i.e., the linear movement axis 21 as well as the rotation axis 22. A horizontal linear movement cannot be performed by the imaging unit 24.

As also shown in FIG. 2, the structural protective barrier 23 in the embodiment has two wall sections 27 and 28. The first wall section 27 is arranged movably and can release a passage 26 which is visible in FIG. 8.

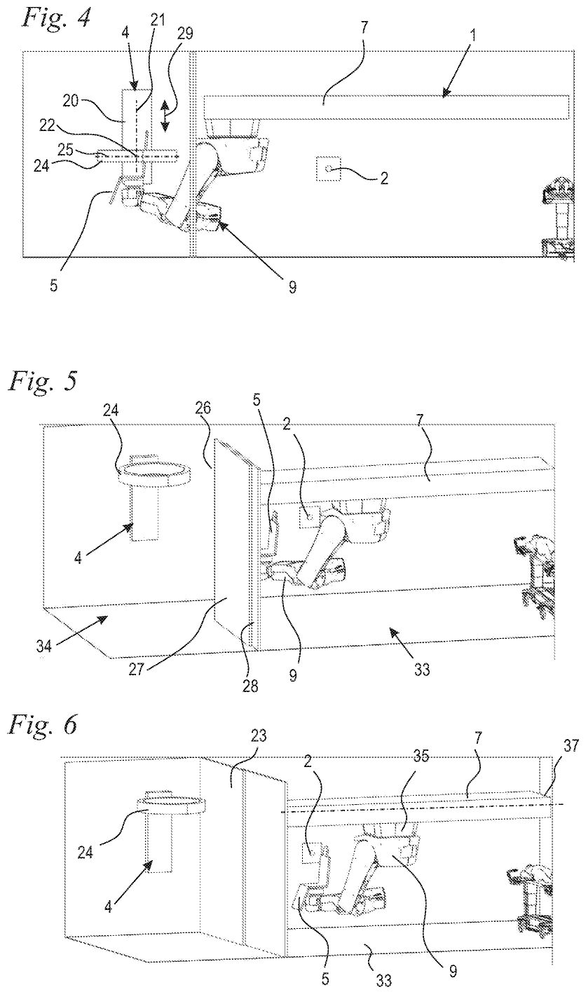

The side view illustrated in FIG. 3 shows the passage 26 (FIG. 8) in open position. The robot arm 6 projects through the passage 26. The patient receptacle 5 is arranged at the imaging device 4 in the second room section 34. In the embodiment, the support 35 of the robot arm 9 in this position is near a first end 36 of the linear movement device 7. The first end 36 is the end of the linear movement device 7 which is facing the structural protective barrier 23. In the illustrated position of FIG. 3, imaging of a patient, not illustrated, positioned on the patient receptacle 5 can be performed by the imaging device 4 to produce an image. For this purpose, the imaging unit 24, as illustrated in FIG. 4, is moved downward across the patient and subsequently, as indicated by the double arrow 29, is moved back upwardly. The imaging unit 24 is in a position in which the imaging plane 25 extends horizontally.

When the imaging unit 24 is moved out of the range of the patient, the patient can be moved out of the second room section 34 through the passage 26 back into the first room section 33. FIG. 5 shows the arrangement when the patient receptacle 5 is located again in the first room section 33. The wall section 27 moves back again into its initial position in order to close the passage 26. Subsequently or while this is done, the patient is positioned in the room section 33 at the radiation source 2. Positioning is realized based on the image of the tissue to be radiated that has been generated by the imaging device 4. For positioning the patient at the radiation source 2, the support 35 of the robot arm 9 is moved by the linear movement device 7 in the direction of the movement axis 8 from the first end 36 in the direction toward a second end 37 of the linear movement device 7. With the structural protective barrier 23 closed, the radiation treatment of the patient is carried out. Since the structural protective barrier 23 is closed, the imaging device 4 is protected from radiation. From the time of generating the image to the end of the radiation treatment, the patient is preferably immobile on the patient receptacle so that a precise positioning is possible.

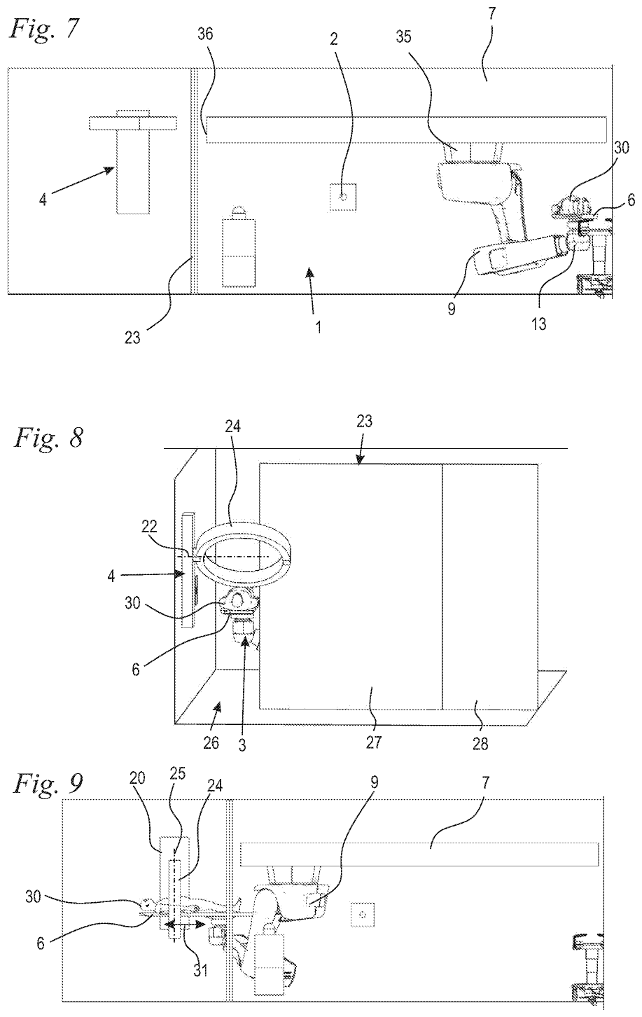

Instead of a seated patient, the patient radiation device 1 can also be used for treating a patient who is in a lying position. In FIG. 7, schematically a patient 30 is illustrated on another patient receptacle 6 which is embodied as a table. The robot arm 9 can pick up the table 6. For this purpose, the arm 13 is attached to the bottom side of the patient receptacle 6. Fastening can be done automatically or manually by an operator. As shown in FIG. 8, subsequently the structural protective barrier 23 is opened by movement of the first wall section 27 and the patient 30 is moved by the patient positioning device 3 to the imaging device 4. In doing so, the support 35 moves along the linear movement device 7 in the direction toward the first end 36 (FIG. 7). In order to carry out imaging of a patient 30 in a lying position, the imaging unit 24 is rotated about the axis of rotation 22 by 90° so that the imaging plane 25 extends vertically. This is illustrated schematically in FIG. 8. The imaging unit 24 can also be moved to a desired height. Subsequently, the patient 30 is moved in horizontal direction relative to the imaging unit 24. The horizontal movement of the patient 30 is realized by the robot arm 9 and/or by the linear movement device 7. A horizontal movement of the imaging unit 24 is not provided. During imaging, the imaging unit 24 is standing still.

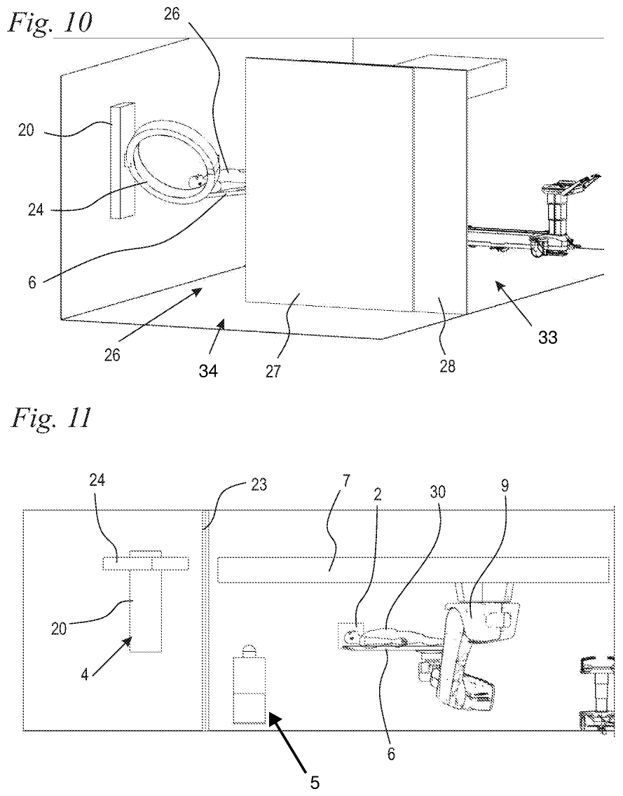

After producing the image, the patient 30 is moved, as indicated schematically in FIG. 10, through the passage 26 into the first room section 33. This movement can be realized advantageously by a combined movement of the robot arm 9 and the linear movement device 7. Subsequently, the structural protective barrier 23 is closed by movement of the first wall section 27 so that the imaging device 4 is protected from radiation. The patient 30 is positioned at the radiation source 2, as illustrated in FIG. 11, and the radiation treatment is carried out.

In an alternative embodiment, it can be provided that the patient positioning device 3 does not comprise a linear movement device. The horizontal movement of the patient receptacle 5, 6 in this embodiment can be carried out advantageously by the robot arm 6. For this purpose, a robot arm 6 is provided that carries out a translatory horizontal movement with sufficient precision by interpolation of rotatory movements.

The specification incorporates by reference the entire disclosure of German priority document 20 2019 001 877.8 having a filing date of Apr. 27, 2019.

While specific embodiments of the invention have been shown and described in detail to illustrate the inventive principles, it will be understood that the invention may be embodied otherwise without departing from such principles.

What is claimed is:

1. A patient radiation device comprising:
a patient positioning device comprising a first patient receptacle, wherein the patient positioning device is configured to carry out at least a horizontal movement of the first patient receptacle;
a radiation source;
an imaging device comprising an imaging unit configured to produce an image in an imaging plane and further comprising a first linear movement device configured to move the imaging unit along a vertical movement axis;
wherein the imaging unit is configured to perform a rotation about a horizontally arranged axis of rotation.

2. The patient radiation device according to claim 1, wherein the patient positioning device comprises a robot arm.

3. The patient radiation device according to claim 2, wherein the robot arm comprises at least six axes of rotation.

4. The patient radiation device according to claim 2, wherein the first patient receptacle is configured to be removably connected to the robot arm.

5. The patient radiation device according to claim 2, wherein the patient positioning device comprises a second patient receptacle, wherein the first and second patient receptacles are configured to be alternatively connected to the robot arm.

6. The patient radiation device according to claim 5, wherein one of the first and second patient receptacles is a seat and the other one of the first and second patient receptacles is a table.

7. The patient radiation device according to claim 2, further comprising a second linear movement device configured to move the robot arm along a horizontal movement axis.

8. The patient radiation device according to claim 7, wherein the second linear movement device is a linear guide system.

9. The patient radiation device according to claim 7, wherein the robot arm is suspended from the second linear movement device.

10. The patient radiation device according to claim 1, further comprising a structural protective barrier configured to separate the imaging device and the radiation source from each other.

11. The patient radiation device according to claim 10, wherein the structural protective barrier is configured to be opened and to form a passage for moving the first patient receptacle from the imaging device to the radiation source.

12. The patient radiation device according to claim 1, wherein the imaging unit is annular.

13. The patient radiation device according to claim 1, wherein the first linear movement device is a linear guide system.

14. The patient radiation device according to claim 1, wherein the imaging unit has only two axes of movement in the form of the vertical movement axis and the horizontally arranged axis of rotation.
